(19) United States
(12) Patent Application Publication
(10) Pub. No.: US 2009/0232724 A1
(43) Pub. Date: Sep. 17, 2009

(54) METHOD OF SEPARATING METALLIC AND SEMICONDUCTING CARBON NANOTUBES FROM A MIXTURE OF SAME

(76) Inventors: Ali Afzali-Ardakani, Ossining, NY (US); James B. Hannon, Mahopac, NY (US); George S. Tulevski, White Plains, NY (US)

Correspondence Address: Vazken Alexanian;IBM CORPORATION, Intellectual Property Law Dept., P.O. Box 218, Yorktown Heights, NY, 10598, US

(21) Appl. No.: 12/045,878

(22) Filed: Mar. 11, 2008

(51) Int. Cl. C01B 31/02; C01B 31/00; C07C 245/20

(52) U.S. Cl. 423/447.2; 423/447.1; 423/414; 534/558; 977/750; 977/752

(57) ABSTRACT

A method which permits large-scale separation of a semiconducting carbon nanotube from a mixture of metallic and semiconducting carbon nanotubes based on differences in solubility resulting from preferentially reacting the metallic carbon nanotubes with an acid functional aryldiazonium salt to form a substantially fully functionalized metallic nanotubes which can be easily separated from the unfunctionalized semiconducting carbon nanotubes.

### BACKGROUND OF THE INVENTION

1. Field of the Invention

The present invention relates to a method of separating metallic carbon nanotubes and semiconducting nanotubes from a mixture of semiconducting and metallic carbon nanotubes. More particularly, the present invention relates to methods of separating metallic carbon nanotubes from semiconducting carbon nanotubes from a mixture thereof using an acid functional diazonium compound.

2. Description of the Related Art

Single walled carbon nanotubes (SWCNTs) have gained enormous interest due to their superior electrical properties. Developing a large-scale, robust processing method for separating semiconducting carbon nanotubes from metallic carbon nanotubes remains a major hurdle in the preparation of integrated electronic circuits. Several approaches to separation according to the type of carbon nanotubes have been reported in the literature.

One approach exploits the subtle differences in the densities of SWCNT as a basis for the separation. This method does not produce a quantitative separation (100%) of SWCNTs, further is limited because the amount of material it can separate is small.

Another approach is common reaction based, which uses an electron acceptor (diazonium salt) to selectively react with metallic carbon nanotubes. The rate in this reaction is much faster for metallic SWCNTs, therefore by controlling the amount and the rate of addition of diazonium compounds it is possible to selectively functionalize metallic nanotubes. So far most of the diazonium compounds that have been used do not render solvent soluble functionalized metallic carbon nanotubes, thus making it extremely difficult to separate the two types of tubes in a large-scale process. Therefore, current methods do not produce physical separation of metallic and semiconducting carbon nanotubes. Thus, a method is needed to provide physical separation of metallic and semiconducting carbon nanotubes. A desirable method is to separate semiconducting carbon nanotubes from a mixture of metallic and semiconducting carbon nanotubes via differences in solubility provided by selectively functionalizing the metallic carbon nanotubes. Accordingly, it is the object of this invention to selectively functionalize metallic SWCNTs with a solubility promoting group such as a hydroxamic acid group, to produce a solvent soluble hydroxamic acid-functionalized SWCNTs, which can be easily separated from unfunctionalized semiconducting SWCNTs. Therefore the functionalized metallic carbon nanotubes can then be converted into non-functionalized metallic carbon nanotubes by heating from about 300° C. to about 600° C. Thus, the present invention provides a method which permits large-scale separation of semiconducting and metallic carbon nanotubes from a mixture of both.

### SUMMARY OF THE INVENTION

The present invention provides a method for separating a semiconducting carbon nanotube from a mixture of metallic and semiconducting carbon nanotubes.

The method includes the steps of:

contacting an acid functional aryldiazonium salt and a room temperature suspension of the mixture of metallic and semiconducting carbon nanotubes in an aqueous solvent; wherein the contacting is carried out at a temperature and for a period of time sufficient to produce a mixture which includes substantially fully functionalized metallic carbon nanotubes and substantially unchanged semiconducting carbon nanotubes;

contacting the mixture that includes substantially fully functionalized metallic carbon nanotubes and substantially unchanged semiconducting carbon nanotubes and at least five equivalents of acetone to precipitate both the functionalized metallic and the unchanged semiconducting carbon nanotubes as a solid mixture;

isolating the solid mixture of functionalized metallic and unchanged semiconducting carbon nanotubes by centrifugation;

purifying the solid mixture of functionalized metallic and unchanged semiconducting nanotubes to remove any surfactant;

sonicating the purified solid mixture of functionalized metallic and unchanged semiconducting carbon nanotubes in a medium to selectively dissolve the functionalized metallic carbon nanotubes; and

separating the unchanged semiconducting carbon nanotubes from the medium by centrifugation.

The present invention further provides a method for separating a semiconducting carbon nanotube from a mixture of metallic and semiconducting carbon nanotubes using a hydroxamic acid functional aryldiazonium salt.

This method includes the steps of:

contacting a hydroxamic acid functional aryldiazonium salt and a room temperature suspension of the mixture of metallic and semiconducting carbon nanotubes in an aqueous solvent; wherein the contacting is carried out at a temperature and for a period of time sufficient to produce a mixture which includes substantially fully functionalized metallic carbon nanotubes and substantially unchanged semiconducting carbon nanotubes;

contacting the mixture that includes substantially fully functionalized metallic carbon nanotubes and substantially unchanged semiconducting carbon nanotubes and at least five equivalents of acetone to precipitate both the functionalized metallic and the unchanged semiconducting carbon nanotubes as a solid mixture;

isolating the solid mixture of functionalized metallic and unchanged semiconducting carbon nanotubes by centrifugation;

purifying the solid mixture of functionalized metallic and unchanged semiconducting nanotubes to remove any surfactant present;

sonicating the solid mixture of functionalized metallic and unchanged semiconducting carbon nanotubes in a medium to selectively dissolve the functionalized metallic carbon nanotubes; and

separating the unchanged semiconducting carbon nanotubes from the medium by centrifugation.

The present invention still further provides hydroxamic acid functional aryldiazonium functionalized single or double walled metallic carbon nanotubes having a purity of at least 95 wt %.

Further still, the present invention provides semiconducting single or double walled carbon nanotubes having at least 95 wt % purity.

An alternative method for separating functionalized metallic carbon nanotubes from unfunctionalized semiconducting carbon nanotubes using dialysis is also provided. This method removes surfactant from the unfunctionalized semiconducting carbon nanotubes using a semi-permeable membrane, leaving the semiconducting carbon nanotubes insoluble in the aqueous medium.

The present invention has the advantages of:

(1) using a solvent soluble hydroxamic acid-functionalized SWCNTs, which can be easily separated from the unfunctionalized semiconducting SWCNTs;

(2) permitting the recovery of the non-functionalized metallic carbon nanotubes by heating functionalized metallic carbon nanotubes at about 300° C. to about 600° C.; and

(3) providing a method which permits large-scale separation of semiconducting and metallic carbon nanotubes from a mixture of both.

### BRIEF DESCRIPTION OF THE DRAWINGS

FIG. 1 is an absorption spectrum depicting reduction of intensity of the peak assigned to the metallic carbon nanotube absorption in which reduced intensity indicates substantial completion of the reaction of metallic carbon nanotubes with an aryldiazonium salt forming a fully functionalized metallic carbon nanotube.

### DETAILED DESCRIPTION OF THE PREFERRED EMBODIMENTS

The method of the present invention is based on differences in solubility. In a preferred embodiment, the method includes the steps of contacting an acid functional aryldiazonium salt and a room temperature suspension of the mixture of metallic and semiconducting carbon nanotubes in an aqueous solvent.

The step is carried out at a temperature and for a period of time sufficient to produce a mixture which includes substantially fully functionalized metallic carbon nanotubes and substantially unchanged semiconducting carbon nanotubes.

Below is a reaction scheme showing the step of contacting a mixture of carbon nanotubes with an aryldiazonium salt to preferentially form functionalized metallic carbon nanotubes.

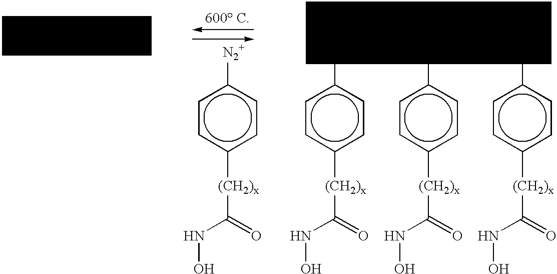

In a preferred embodiment the suitable aryldiazonium includes an aryldiazonium cation represented by the formulas:

⁺N₂—Ar—(CH₂)ₙCONHOH,

⁺N₂—Ar—(CH₂)ₙCOOH,

⁺N₂—Ar—(CH₂)ₙSO₂OH,

⁺N₂—Ar—(CH₂)ₙP(O)₂OH,

⁺N₂—Ar—CONHOH,

⁺N₂—Ar—SO₂NHOH,

⁺N₂—Ar—P(O)₂NHOH,

⁺N₂—Ar—COOH,

⁺N₂—Ar—SO₃H, and

⁺N₂—Ar—PO₃H;

wherein Ar is a substituted or unsubstituted aromatic group having one or more substituents each independently selected from alkyl of 1 to 4 carbon atoms, halogen, or alkoxy; and wherein n is 0 to 4.

In another embodiment for forming soluble derivatized metallic single walled carbon nanotubes for separation from a mixture of single walled carbon nanotube types, both the sulfonic acid and the phosphonic acid functionalized aryldiazonium salts are substituted for the hyroxamic acid functionalized diazonium salt.

A sulfonic acid functionalized aryldiazonium salt can be used to obtain functionalized metallic single walled carbon nanotubes from the reaction of metallic single walled carbon nanotubes with the sulfonic acid functionalized arlydiazonium salt A.

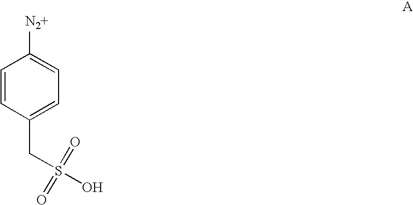

In addition, a phosphonic acid functionalized arlydiazonium salt functionalized metallic single walled carbon nanotubes can be obtained from the reaction of metallic single walled carbon nanotubes with the phosphonic acid functionalized aryldiazonium salt B.

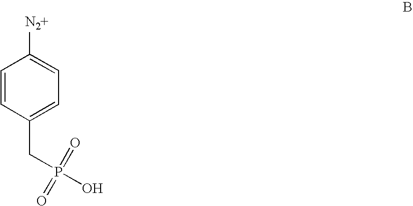

Alternatively carboxcylic acid functional aryldiazonium salt C can be used instead of the hydroxamic acid functional diazonium salts in the reaction with metallic single walled carbon nanotubes.

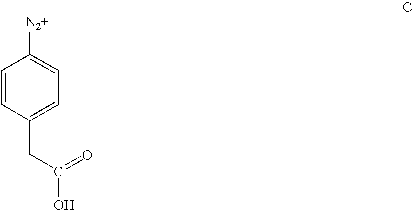

In another embodiment for forming soluble derivatized metallic single walled carbon nanotubes for separation from a mixture of single walled carbon nanotube types, both the sulfonic acid and the phosphonic acid functionalized aryldiazonium salts are substituted for the hyroxamic acid functionalized diazonium salt.

The suspending of the carbon nanotubes in the surfactant component, necessary to suspend the carbon nanotubes in water, is preferably an aqueous solution of sodium cholate, sodium dodecyl sulfate (SDS), Triton-X™, a product of BASF, Philadelphia, Pa., common bile salts, or a mixture thereof. Triton-X™ is a nonionic surfactant having the formula 4-(C₈H₁₇)C₆H₄(OCH₂CH₂)_{N}OH and structural formula:

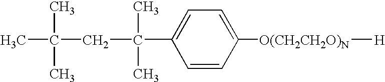

wherein N is from 1 to 10 which correspond to the number of repeat units of 1 to 10.

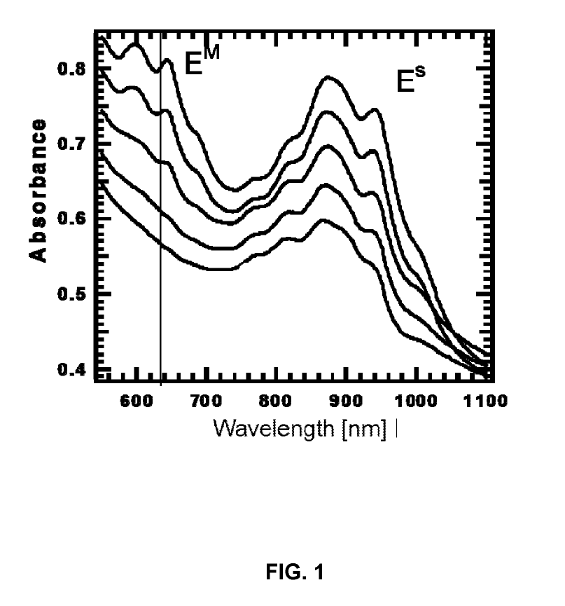

FIG. 1 shows the formation of a covalent bond preferentially between the metallic single walled carbon nanotubes and the hyhroxyamic acid aryldiazonium salt. The attenuation of the metallic peak provides evidence that the reaction is complete. Specifically, the unreacted metallic single walled carbon nanotubes have an absorbance at 450 to 650 wavenumbers of 0.08.

Following the completion of the reaction of the metallic single walled carbon nanotubes with the hydroxamic acid aryldiazonium salt the absorbance reduces to 0.06 at 450-650 wavenumbers. The substantial drop of the metallic transition absorbance demonstrates the completion of the reaction and the bond formation.

The hydroxamic acid aryldiazonium salt solution is added to the suspension of mixture of single walled carbon nanotubes at a rate of 1 ml/hr.

Alternatively, dichloromethane is contacted to the mixture of metallic and semiconducting single walled carbon nanotubes prior to reaction with the hydroxyamic acid aryldiazonium salt solution.

The suspension of functionalized metallic and unchanged semiconducting carbon nanotubes is then purified to remove any surfactant by suspending the solid mixture of functionalized metallic and unchanged semiconducting nanotubes in water followed by centrifugation carried out at a rate of 15 KRPM for about 20 minutes. The step of purifying to remove surfactant may optionally be repeated at least once. Removing surfactant is followed by contacting the mixture of substantially fully functionalized metallic carbon nanotubes and substantially unchanged semiconducting carbon nanotubes with at least five equivalents of acetone to precipitate a solid mixture of substantially fully functionalized metallic carbon nanotubes and substantially unchanged semiconducting carbon nanotubes. The solid mixture of functionalized metallic and unchanged semiconducting carbon nanotubes from the step of precipitation is then isolated from the supernatant liquid by centrifugation.

The purified solid mixture of functionalized metallic and unchanged semiconducting carbon nanotubes is then sonicated for 30 minutes, 600 Watts at 40% maximum power in a medium, such as aqueous base, to selectively dissolve the functionalized metallic carbon nanotubes. Other examples of such media include aqueous bases of alkali metals, ammonium hydroxide, a 1M sodium hydroxide aqueous solution, water or super critical fluid. This step of sonicating in the medium to dissolve the functionalized metallic carbon nanotubes has a selectivity of at least 85%.

Unchanged semiconducting carbon nanotubes are then separated from the medium containing functionalized metallic carbon nanotubes by centrifugation. Centrifugation can performed at low speed. The semiconducting carbon nanotubes are then collected and dried.

In another preferred embodiment in the metallic carbon nanotubes can be isolated and collected by the steps of collecting the medium containing functionalized metallic carbon nanotubes from the separation step, precipitating functionalized metallic carbon nanotubes by contacting the medium with at least 5 equivalents of acetone to precipitate the metallic carbon nanotubes.

In another preferred embodiment, the semiconducting nanotubes can be preferentially precipitated using dialysis. The mixture containing the functionalizing metallic carbon nanotubes and surfactant wrapped semiconducting nanotubes are placed in a dialysis vessel with a semi-permeable membrane that allows molecules less than 10 k Daltons to pass through. The vessel is then placed in a water bath and allowed to undergo dialysis for 12 hours. The surfactant will then diffuse out of the vessel via mass transfer driving the concentration of surfactant down until the semiconducting carbon nanotubes are forced to precipitate out of solution. The metallic tubes are covalently linked to compounds that allow them to stay soluble in solution.

The step of recovering the metallic carbon nanotubes is by annealing the functionalized metallic carbon nanotubes at about 300° C. to about 600° C. for 120 seconds in an inert atmosphere, such as argon, to produce the metallic carbon nanotubes, i.e., the pure de-functionalized metallic single walled carbon nanotubes.

Any suitable carbon nanotube can be utilized in the present invention. For example, suitable carbon nanotubes can be single walled carbon nanotubes, double walled carbon nanotubes, multiwalled carbon nanotubes or a combination thereof. Suitable carbon nanotube can be obtained by any method of production, such as, for example, laser-ablation, HiPCo, CoMoCat and arc-discharge. Such laser-ablation and HiPCo derived carbon nanotubes are available from Carbon Nanotechnologies Inc., 16200 Park Row Houston, Tex. 77084.

In an another preferred embodiment of the present invention the steps of the method for separating a semiconducting carbon nanotube from a mixture of metallic and semiconducting carbon nanotubes include contacting a hydroxamic acid functional aryldiazonium salt and a room temperature suspension of a mixture of metallic and semiconducting carbon nanotubes in an aqueous solvent. Contacting is carried out at a temperature and for a period of time sufficient to produce a mixture which includes substantially fully functionalized metallic carbon nanotubes and substantially unchanged semiconducting carbon nanotubes.

The solid mixture of functionalized metallic and unchanged semiconducting carbon nanotubes is then contacted with water and centrifuged to remove any surfactant. This step of purifying the solid mixture of functionalized metallic and unchanged semiconducting carbon nanotubes to remove surfactant is followed by contacting the mixture of substantially fully functionalized metallic carbon nanotubes and substantially unchanged semiconducting carbon nanotubes with at least five equivalents of acetone to precipitate both the functionalized metallic and the unchanged semiconducting carbon nanotubes as a solid mixture.

The solid mixture is then sonicated in a medium to selectively dissolve functionalized metallic carbon nanotubes, thus separating unchanged semiconducting carbon nanotubes from the medium as the semiconducting carbon nanotubes form a precipitate. Centrifugation is then used to facilitate further separation of the precipitate from the supernantant liquid. This process yields semiconducting single or double walled carbon nanotubes with at least 95% wt purity.

Also, the method of isolation and collecting hydroxamic acid functional aryldiazonium functionalized single or double walled metallic carbon nanotubes has a purity of at least 95 wt %.

In another preferred embodiment of the invention the method utilizing hydroxamic acid functional metallic carbon nanotubes can also be used to isolate and purify metallic nanotubes from a mixture of semiconducting and metallic carbon nanotubes. The reaction that preferentially forms the functionalized metallic carbon nanotubes is monitored spectroscopically. Observation of the attenuation of the metallic absorption peak from 450 to 650 wavenumbers is evidence of the formation of hydroxamic acid functional arlydiazonium functionalized metallic carbon nanotubes with a purity of at least 95 wt %. Once the medium containing functionalized Metallic carbon nanotubes is isolated from the solid unchanged semiconducting carbon nanotubes by precipitating with acetone, the solid hydoxamic acid functional aryldiazonium functionalized metallic carbon nanotubes can then yield single or double walled metallic carbon nanotubes when subjected to an inert atmosphere at about 300° C. to about 600° C.

Metallic carbon nanotube with a purity of at least 95 wt % result from heating in an inert atmosphere. In this heating step the hydroxamic aryldiazonium moiety is removed.

One skilled in the art would appreciate any one of the arlydiazonium used to functionalize metallic carbon nanotubes can be removed by the heating of any of the aryldiazonium functionalized metallic carbon nanotubes at about 300° C. to about 600° C. in an inert atmosphere, yielding metallic carbon nanotubes at a purity of at least 95%.

The preferred embodiment is a method that includes contacting an acid functional aryldiazonium salt in a solvent of 1:1 methanol:water and suspension of the mixture of metallic and semiconducting carbon nanotubes in an aqueous solvent. Alternatively, dichloromethane is substituted for the 1:1 methanol/water solution. Preferably, the contacting can be carried out at about zero to about 25° C. at rate of 1 mL/hr or a period of time sufficient to produce a mixture which includes substantially fully functionalized metallic carbon nanotubes and substantially unchanged semiconducting carbon nanotubes.

The above reaction can be monitored by absorption spectroscopy as illustrated by FIG. 1. Referring to FIG. 1, the absorption spectrum of the step of contacting the acid functional aryldiazonium salt and a mixture of metallic and semiconducting carbon nanotubes in an aqueous solvent is shown. A reduction in absorption intensity in an amount of 0.08 at 650 wavenumbers for the metallic transition peak to an intensity of about 0.057 at 650 wavenumbers is evidence for formation of the functionalized metallic carbon nanotube and substantial completion of the reaction.

### EXAMPLE

### A. Preparation of Hydroxamic Acid Aryidiazonium Chloride:

Oxalyl chloride (10.16 g, 0.08 mole) was added to a solution of 4-nitrobenzoic acid (6.68 g, 0.04 mole) in 100 mL of anhydrous dichloromethane. A drop of N,N-dimethylformamide was added and the mixture was stirred under nitrogen for 3 hours. The solvent and excess oxalyl chloride was evaporated under reduced pressure. The residual oily compound was redissolved in 20 mL of anhydrous methanol and added to a solution of o-benzylhydroxylamine hydrochloride (0.04 mole) and triethylamine (0.08 mole) in 50 mL of anhydrous dichloromethane. The mixture was stirred at room temperature for 4 hours and then washed with dilute hydrochloric acid and brine, dried over anhydrous magnesium sulfate and filtered. Evaporation of the solvent gave light brown solid of the formula:

para-⁺N₂—C₆H₄—(CH₂)ₙCONHOH Cl⁻

The isolated brown solid was crystallized from ethanol to produce O-benzyl-4-nitrophenylhydroxamic acid as white crystals. Palladium on activated carbon (10%, 500 mg) was added to a mixture of O-benzyl-4-nitrophenylhydroxamic acid (2.71 g, 0.01 mole) and ammonium formate (3.65 g, 0.05 mole) in 100 mL anhydrous methanol and the solution was refluxed under nitrogen for 4 hours. The hot mixture was then filtered and the solvent was removed under reduced pressure. The residual solid was crystallized from water to give 4-aminophenyl hydroxamic acid. Then, nitrosonium tetrafluoroborate (118 mg, 1 mmole) was added to a suspension of 4-aminophenylhydroxamic acid (152 mg, 1 mmole) in 10 mL anhydrous acetonitrile and the solution was stirred under nitrogen for 30 minutes to produce a yellow diazonium salt. The reagents used in the synthesis the hydroxamic acid aryldiazonium salt were obtained from the Sigma-Aldrich, Milwaukee, Wis.

### B. Preparation of Suspension of SWCNT Mixture:

A concentration of 10 mg of laser ablation grown SWCNTs are added to 10 ml of 2% sodium cholate w/v in purified water. The mixture is sonicated for 1 hour with a 600 W horn sonicator operated at 35% power. The resultant solution is then added to a centrifuge tube and then centrifuged for 20 minutes at 25,000 RPM. A step gradient is then formed by first added 5 ml of the SWCNT solution into a 10 ml centrifuge tube. Five ml of a 60% solution of lodixinol in water is added via syringe to the centrifuge tube and since it has a much higher density than the SWCNT solution, it will settle to the bottom of the tube making a separate phase. The centrifuge tube is then centrifuged for 15 hours at 41,000 rpm and thereafter removed from the rotor and the SWCNTs accumulate at the interface of the two phases whereupon it is removed with a pipette.

### C. Selective Functionalization of Metallic Singlewalled Carbon Nanotubes:

Two ml of the purified SWCNT suspension was diluted 1:10 in purified water. The diazonium salt solution from part B above was added dropwise via a syringe pump at a rate of 1 ml/hour. After every 100 ml of solution added, an aliquot was taken and added to a cuvette where a uv-vis spectra was taken to measure the intensity of the metallic peak (400-650 nm). This was done repeatedly until the metallic peak completely disappeared (approximately 600 ml of solution was needed). The solution was then diluted by adding 5 equivalents of acetone to precipitate the SWCNTs. The precipitate was collected via centrifugation at 3,000 RPM for five minutes. A small amount of deionized water was added to the solid and then sonicated in a bath sonicator for 30 minutes. The last two steps were repeated to produce a precipitate of a mixture of purified functionalized metallic nanotubes and unfunctionalized semiconducting nanotubes. Deionized water was added to the mixture to dissolve the functionalized metallic nanotubes leaving behind the desired semiconducting nanotubes as a pure solid.

### D. Regeneration of Metallic Carbon Nanotubes From Functionalized Metallic Carbon Nanotubes:

The metallic carbon nanotubes can be easily regenerated by heating the functionalized metallic carbon nanotubes at from about 300° C. to about 600° C. in an inert atmosphere of Argon for 2 minutes hours at atmospheric pressure in a any vessel capable of heating to the required temperature in an inert atmosphere, such as, a rapid thermal annealer or a tube furnace, to produce the metallic carbon nanotubes according to the method of the present invention which permits large-scale separation of semiconducting and metallic carbon nanotubes from a mixture thereof.

The present invention has been described with particular reference to the preferred embodiments. It should be understood that variations and modifications thereof can be devised by those skilled in the art without departing from the spirit and scope of the present invention. Accordingly, the present invention embraces all such alternatives, modifications and variations that fall within the scope of the appended claims.

We claim:

1. A method for separating a semiconducting carbon nanotube from a mixture of metallic and semiconducting carbon nanotubes, said method comprising:
contacting an acid functional aryldiazonium salt and a room temperature suspension of said mixture of metallic and semiconducting carbon nanotubes in an aqueous solvent; wherein said contacting is carried out at a temperature and for a period of time sufficient to produce a mixture which includes substantially fully functionalized metallic carbon nanotubes and substantially unchanged semiconducting carbon nanotubes;
contacting said mixture which includes substantially fully functionalized metallic carbon nanotubes and substantially unchanged semiconducting carbon nanotubes and at least five equivalents of acetone to precipitate both said functionalized metallic and said unchanged semiconducting carbon nanotubes as a solid mixture;
isolating said solid mixture of functionalized metallic and unchanged semiconducting carbon nanotubes by centrifugation;
purifying said solid mixture of functionalized metallic and unchanged semiconducting nanotubes to remove any surfactant;
sonicating said purified solid mixture of functionalized metallic and unchanged semiconducting carbon nanotubes in a medium to selectively dissolve said functionalized metallic carbon nanotubes; and
separating said unchanged semiconducting carbon nanotubes from said medium by centrifugation.

2. The method of claim 1, wherein said aryldiazonium salt is selected from the group consisting of:
a hydroxamic acid functional aryldiazonium salt, a carboxylic acid functional aryldiazonium salt, a sulfonic acid functional aryldiazonium salt, and a phosphoric acid functional aryldiazonium salt.

3. The method of claim 1, wherein said aryldiazonium is selected from the group consisting of compounds represented by the formula:
⁺N₂—Ar—(CH₂)ₙCONHOH,
⁺N₂—Ar—(CH₂)ₙCOOH,
⁺N₂—Ar—(CH₂)ₙSO₂OH,
⁺N₂—Ar—(CH₂)ₙP(O)₂OH,
⁺N₂—Ar—CONHOH,
⁺N₂—Ar—O₂NHOH,
⁺N₂—Ar—P(O)₂NHOH,
⁺N₂—Ar—COOH,
⁺N₂—Ar—SO₃H, and
⁺N₂—Ar—PO₃H;
wherein Ar is a substituted or unsubstituted aromatic group having one or more substituents each independently selected from the group consisting of: alkyl of 1 to 4 carbon atoms, halogen, and alkoxy; and
wherein n is 0 to 4.

4. The method of claim 3, wherein said hydroxamic acid functional aryldiazonium is represented by the formula:
para-⁺N₂—C₆H₄—(CH₂)ₙCONHOH

5. The method of claim 4, wherein n is 1.

6. The method of claim 1, wherein formation of said functionalized metallic carbon nanotube is monitored using absorption spectroscopy to determine the point of substantial completion.

7. The method of claim 1, wherein said centrifugation is carried out at a rate of 15 KRPM for about 20 minutes.

8. The method of claim 1, wherein said isolation is carried out by dialysis.

9. The method of claim 1, wherein said aqueous solvent is a 1:1 methanol:water solvent.

10. The method of claim 1, wherein said aryldiazonium salt solution and said aqueous solvent are first contacted to form a 0.1 mM solution.

11. The method of claim 10, wherein said aryldiazonium salt solution is added to said suspension of said mixture of carbon nanotubes at a rate of 1 ml/hr.

12. The method of claim 10, wherein said surfactant is selected from the group consisting of: sodium cholate, sodium dodecyl sulfate, 4-(C₈H₁₇)C₆H₄(OCH₂CH₂)ₙOH wherein n=10, and common bile salts, and a mixture thereof.

13. The method of claim 1, where said suspension further comprises a surfactant.

14. The method of claim 1, wherein said aqueous solvent further comprises a base.

15. The method of claim 1, wherein said medium is selected from the group consisting of: water, aqueous base, and a super critical fluid.

16. The method of claim 1, wherein said solid mixture is sonicated for 30 minutes at room temperature.

17. The method of claim 1, wherein the step of purifying is carried out by a method comprising:
suspending said solid mixture of functionalized metallic and unchanged semiconducting nanotubes in water;
isolating said solid mixture of functionalized metallic and unchanged semiconducting carbon nanotubes by centrifugation; and optionally repeating these steps at least once.

18. The method of claim 1, further comprising:
collecting said medium containing functionalized metallic carbon nanotubes from the separation step;
precipitating said functionalized metallic carbon nanotubes by contacting said medium and at least 5 equivalents of acetone to precipitate said metallic carbon nanotubes;
collecting said metallic carbon nanotubes;
heating said metallic carbon nanotubes at about 300° C. to about 600° C. in an inert atmosphere to recover the metallic carbon nanotubes.

19. The method of claim 1, wherein the step of sonicating in said medium to dissolve said functionalized metallic carbon nanotubes has a selectivity of at least 85%.

20. The method of claim 1, wherein said carbon nanotubes are selected from the group consisting of:
single walled carbon nanotubes, double walled carbon nanotubes, and a combination thereof.

21. A method for separating a semiconducting carbon nanotube from a mixture of metallic and semiconducting carbon nanotubes, said method comprising:
contacting a hydroxamic acid functional aryldiazonium salt and a room temperature suspension of said mixture of metallic and semiconducting carbon nanotubes in an aqueous solvent; wherein said contacting is carried out at a temperature and for a period of time sufficient to produce a mixture which includes substantially fully functionalized metallic carbon nanotubes and substantially unchanged semiconducting carbon nanotubes;
contacting said mixture which includes substantially fully functionalized metallic carbon nanotubes and substantially unchanged semiconducting carbon nanotubes and at least five equivalents of acetone to precipitate both said functionalized metallic and said unchanged semiconducting carbon nanotubes as a solid mixture;
isolating said solid mixture of functionalized metallic and unchanged semiconducting carbon nanotubes by centrifugation;
purifying said solid mixture of functionalized metallic and unchanged semiconducting nanotubes to remove any surfactant present;
sonicating said solid mixture of functionalized metallic and unchanged semiconducting carbon nanotubes in a medium to selectively dissolve said functionalized metallic carbon nanotubes; and
separating said unchanged semiconducting carbon nanotubes from said medium by centrifugation.

22. The method of claim 21, wherein said functionalized metallic carbon nanotube is monitored using absorption spectroscopy to determine the point of substantial completion.

23. The method of claim 21, wherein said centrifugation is carried out at a rate of 15 KRPM for about 20 minutes.

24. The method of claim 21, wherein said isolation is by dialysis.

25. The method of claim 21, wherein said aqueous solvent is a 1:1 methanol:water solvent.

26. The method of claim 21, wherein said hydroxamic acid functional aryldiazonium salt solution and said aqueous solvent are first contacted to form a 0.1 mM solution.

27. The method of claim 21, wherein said hydroxamic acid functional aryldiazonium salt solution is added to the suspension of said mixture of carbon nanotubes at a rate of 1 ml/hr.

28. The method of claim 21, where said suspension further comprises a surfactant.

29. The method of claim 28, wherein said surfactant is selected from the group consisting of: sodium cholate, sodium dodecyl sulfate, 4-(C₈H₁₇)C₆H₄(OCH₂CH₂)ₙOH wherein n=10, and common bile salts, and a mixture thereof.

30. The method of claim 21, wherein said aqueous solvent further comprises a base.

31. The method of claim 30, wherein said base is 1M sodium hydroxide.

32. The method of claim 21, wherein said medium is selected from the group consisting of: water, aqueous base, and a super critical fluid.

33. The method of claim 32, wherein said solid mixture is sonicated for 30 minutes at room temperature.

34. The method of claim 21, wherein said hydroxamic acid functional aryldiazonium is represented by the formula:
⁺N₂—Ar—(CH₂)ₙCONHOH
wherein Ar is a substituted or unsubstituted aromatic group;
wherein each substituent in said substituted aromatic group is independently selected from the group consisting of: alkyl of 1-4 carbon atoms, halogen, and alkoxy; and
wherein n is 0 to 4.

35. The method of claim 34, wherein said hydroxamic acid functional aryldiazonium is represented by the formula:
para-⁺N₂—C₆H₄—CH₂CONHOH

36. The method of claim 21, wherein the step of purifying is carried out by a method comprising:
suspending said solid mixture of functionalized metallic and unchanged semiconducting nanotubes in water; and
isolating said solid mixture of functionalized metallic and unchanged semiconducting carbon nanotubes by centrifugation; and optionally
repeating these steps at least once.

37. The method of claim 21, further comprising:
collecting said medium containing functionalized metallic carbon nanotubes from the separation step;
precipitating said functionalized metallic carbon nanotubes by contacting said medium and at least 5 equivalents of acetone to precipitate said metallic carbon nanotubes;
collecting said metallic carbon nanotubes;
heating said metallic carbon nanotubes at about 300° C. to about 600° C. in an inert atmosphere to recover the metallic carbon nanotubes.

38. A hydroxamic acid functional aryldiazonium functionalized single or double walled metallic carbon nanotubes having a purity of at least 95 wt %.

39. A semiconducting single or double walled carbon nanotube having at least 95 wt % purity.
